(19) United States
(12) Patent Application Publication
(10) Pub. No.: US 2022/0183546 A1
(43) Pub. Date: Jun. 16, 2022

(54) AUTOMATED VISION TESTS AND ASSOCIATED SYSTEMS AND METHODS

(71) Applicants: William V. Padula, Killingworth, CT (US); Ted Dinsmore, Killingworth, CT (US); Chris Andrews, Granger, IN (US); Craig Andrews, Loudon, TN (US)

(72) Inventors: William V. Padula, Killingworth, CT (US); Ted Dinsmore, Killingworth, CT (US); Chris Andrews, Granger, IN (US); Craig Andrews, Loudon, TN (US)

(21) Appl. No.: 17/117,227

(22) Filed: Dec. 10, 2020

(51) Int. Cl. A61B 3/00; G06N 20/00; A61B 3/113; A61B 3/032; A61B 3/09; A61B 3/06; A61B 3/107; A61B 3/11; A61B 3/024

(52) U.S. Cl. CPC A61B 3/005; G06N 20/00; A61B 3/0025; A61B 3/113; A61B 3/032; A61B 3/024; A61B 3/066; A61B 3/107; A61B 3/112; A61B 3/0033; A61B 3/09

(57) ABSTRACT

A system and method for conducting automated vision tests and associated training using artificial intelligence processing on an extended reality (XR) platform includes: an extended reality headset display device configured to be worn by a user and operated by the user without direct medical professional assistance; a computing device communicatively coupled to the extended reality headset display device; and a vision testing and training module configured to execute on the computing device, the vision testing module when executed: displays at least one test data set comprising a plurality of vision tests to a user; detects a plurality of user responses to the tests; records the plurality of user responses; processes the plurality of user responses; and stores the plurality of user responses to compare with a plurality of other recorded user data to determine standards based on user qualifications.

### FIELD OF THE INVENTION

The technology described herein relates generally to methods, systems, and devices for the testing of human subjects for a multiplicity of vision tests and for vision training. More specifically, this technology relates to an automated virtual assistant and eye-movement recording device with extended reality, augmented reality, and virtual reality platforms for automated vision tests of saccades/pursuits, visual acuity, fixations, regressions, depth perception, convergence, divergence, color tests, speed, Amsler grid, keratometry, pupillometry, colorimetry, and other field tests. Furthermore, this technology relates to testing and assessment devices, extended reality, augmented reality, and virtual reality goggles, headsets, motion-sensing cameras, and vision training devices.

### BACKGROUND OF THE INVENTION

It is known the in the background art that doctors have provided eye examinations to conduct various vision tests. Many doctors use trained professional assistants to conduct preliminary tests prior to seeing the patients themselves. Such vision tests, for example, may include one or more of visual acuities, gross fields, depth perception, color vision, and saccades/pursuits. Often such tests are conducted in a preliminary screening room or in the exam room prior to the doctor seeing the patient. It is expensive to train and maintain professional vision assistants to conduct these various vision tests.

Additionally, recorders for tracking eye movements are known in the background art and have been available for approximately a century. For example, early models included video cameras but required data collection with pen and paper. Over time, such devices evolved to include infrared technology and later computer databases accessible over the internet. However, these known systems have many shortcomings.

Related utility patents known in the art include the following:

U.S. Pat. No. 7,367,675, issued to Maddalena et al. on May 6, 2008, discloses a vision testing system. Specifically, a method and apparatus are provided for testing the vision of a human subject using a series of eye tests. A test setup procedure is run to adjust the settings of a display device such that graphic objects displayed on the device conform to a pre-defined appearance. A series of preliminary tests, static tests and dynamic tests are displayed on the device, and the responses of the subject are recorded. The tests may be run remotely, for example over the Internet. No lenses are required to run the tests.

Related patent application publications known in the art include the following:

U.S. Patent Application Publication No. 2019/0261847 filed by Padula et al. and published on Aug. 29, 2019, discloses a holographic real space refractive sequence, and which is incorporated herein by reference. Specifically, a system and a method for holographic refraction eye testing device is disclosed. The system renders one or more three dimensional objects within the holographic display device. The system updates the rendering of the one or more three dimensional objects within the holographic display device, by virtual movement of the one or more three dimensional objects within the level of depth. The system receives input from a user indicating alignment of the one or more three dimensional objects after the virtual movement. The system determines a delta between a relative virtual position of the one or more three dimensional objects at the moment of receiving input and an optimal virtual position and generates prescriptive remedy based on the delta.

Related non-patent literature known in the art includes the following:

RightEye has disclosed some basic eye movement recorder technology. RightEye is available online at this site, www.righteye.com.

Known systems and methods for vision tests and eye movement recordation are inadequate. Others have attempted to overcome these deficiencies with new tests and methods for vision tests and eye movement recordation; however, these tests and methods have been found also to have various shortcomings. These shortcomings are addressed and overcome by the systems and methods of the technology described herein.

The foregoing patent and other information reflect the state of the art of which the inventors are aware and are tendered with a view toward discharging the inventors' acknowledged duty of candor in disclosing information that may be pertinent to the patentability of the technology described herein. It is respectfully stipulated, however, that the foregoing patent and other information do not teach or render obvious, singly or when considered in combination, the inventors' claimed invention.

### BRIEF SUMMARY OF THE INVENTION

In various exemplary embodiments, the technology described herein provides methods, systems, and devices for the testing of human subjects for a multiplicity of vision tests. More specifically, the technology described herein provides an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests. Furthermore, the technology described herein provides testing and assessment devices, extended reality, augmented reality, and virtual reality goggles, headsets, motion-sensing cameras, and vision training devices.

In one exemplary embodiment, the technology described herein provides a system for conducting automated vision tests and associated training using artificial intelligence processing on an extended reality (XR) platform. Based on user test results with the XR platform, as measured and recorded from the automated vision tests and compared with a database of normative standards, an optometrist or ophthalmologist may determine and recommend that the user engage in prescribed training exercises using this XR platform and/or determine and prescribe that other visual therapies are needed. The system includes: an extended reality headset display device configured to be worn by a user and operated by the user without direct medical professional assistance; a computing device communicatively coupled to the extended reality headset display device; and a vision testing and training module configured to execute on the computing device, the vision testing module when executed: displays at least one test data set comprising a plurality of vision tests to a user; detects a plurality of user responses to the tests; records the plurality of user responses; processes the plurality of user responses; and stores the plurality of user responses to compare with a plurality of other recorded user data to determine standards based on user qualifications.

In at least one embodiment of the system, the vision testing and training module further includes a saccades vision testing and training module configured to execute on the computing device, the saccades vision testing module when executed: displays a standardized font set at a standardized distance to display a few paragraphs of text at a specified visual angle to a user; detects a motion of at least one eye of the user in a vertical and a horizontal plane; records a plurality of eye movements of the at least one eye; processes the recorded eye movements to determine a plurality of features of the eye movements; and stores the recorded eye movements to compare with a plurality of other recorded user data to determine standards based on user qualifications.

In at least one embodiment of the system, the vision testing and training module further includes a visual acuity vision testing and training module configured to execute on the computing device, the visual acuity vision testing module when executed: displays at a standardized distance a test data set to comprising a plurality of visual acuity tests and optotypes to a user; detects a plurality of user responses, vocal or virtual, to the visual acuity tests; records the plurality of user responses; processes the plurality of user responses; and stores the plurality of user responses to compare with a plurality of other recorded user data to determine standards based on user qualifications.

In at least one embodiment of the system, the vision testing and training module further includes a gross field vision testing and training module configured to execute on the computing device, the gross field vision testing module when executed: displays at a standardized distance at least one gross field test to a user; detects a user response, vocal or virtual, to the gross field test; records the user response; processes the user response; forwards, if the gross field test result is a fail, the gross field result to indicate a full field test is recommended; and stores the user response to compare with a plurality of other recorded user data to determine standards based on user qualifications.

In at least one embodiment of the system, the vision testing and training module further includes a depth perception vision testing and training module configured to execute on the computing device, the depth perception vision testing module when executed: utilizes right eye and left eye projections in space; displays at a distance of optical infinity and at a reading distance at least one depth perception test to a user; detects a user response, vocal or virtual, to the depth perception vision test; records the user response; processes the user response; and stores the user response to compare with a plurality of other recorded user data to determine standards based on user qualifications.

In at least one embodiment of the system, the vision testing and training module further includes a color vision testing and training module configured to execute on the computing device, the gross field vision testing module when executed: utilizes a plurality of color test projections; displays at a standardized distance at least one color vision test to a user; detects a user response, vocal or virtual, to the color vision test; records the user response; processes the user response; and stores the user response to compare with a plurality of other recorded user data to determine standards based on user qualifications.

In at least one embodiment of the system, the vision testing and training module further includes a speed vision testing and training module configured to execute on the computing device, the speed vision testing module when executed: utilizes a plurality of speed reading tests; displays at a standardized distance at least one speed vision test to a user; detects a user response, vocal or virtual, to the speed vision test; records the user response; processes the user response; and stores the user response to compare with a plurality of other recorded user data to determine standards based on user qualifications.

In at least one embodiment of the system, the vision testing and training module further includes an Amsler grid vision testing and training module configured to execute on the computing device, the Amsler grid vision testing module when executed: utilizes an Amsler grid test; displays at a standardized distance an Amsler grid vision test to a user; detects a user response, vocal or virtual, to the Amsler grid vision test; records the user response; processes the user response; and stores the user response to compare with a plurality of other recorded user data to determine standards based on user qualifications.

In at least one embodiment of the system, the vision testing and training module further includes a keratometry vision testing module configured to execute on the computing device, the keratometry vision testing module when executed: utilizes a keratometry vision test; utilizes a Placido disc image; displays a Placido disc image to a user; determines the curvature characteristics of the anterior surface of the cornea; records the curvature characteristics; processes the curvature characteristics; and stores the curvature characteristics to compare with a plurality of other recorded user data to determine standards based on user qualifications.

In at least one embodiment of the system, the vision testing and training module further includes a pupillometry vision testing module configured to execute on the computing device, the pupillometry vision testing module when executed: utilizes a pupillometry vision test; displays a light to a user; checks the pupil size; measures the pupillary response of the user to the light; records the pupillary response; processes the pupillary response; and stores the pupillary response to compare with a plurality of other recorded user data to determine standards based on user qualifications.

In at least one embodiment of the system, the vision testing and training module further includes a colorimetry vision testing module configured to execute on the computing device, the colorimetry vision testing module when executed: utilizes a colorimetry dynamic and static field vision test; displays a plurality of colored lights to a user; measures the response of the user to the plurality of colored lights; records the response; processes the response; and stores the response to compare with a plurality of other recorded user data to determine standards based on user qualifications.

In another exemplary embodiment, the technology described herein provides a method for conducting automated vision tests and associated training using artificial intelligence processing on an extended reality (XR) platform. Based on user test results with the XR platform, as measured and recorded from the automated vision tests and compared with a database of normative standards, an optometrist or ophthalmologist may determine and recommend that the user engage in prescribed training exercises using this XR platform and/or determine and prescribe that other visual therapies are needed. The method includes: utilizing an extended reality headset display device configured to be worn by a user and operated by the user without direct medical professional assistance; utilizing a computing device communicatively coupled to the extended reality headset display device; utilizing a vision testing and training module configured to execute on the computing device; displaying at least one test data set comprising a plurality of vision tests to a user; detecting a plurality of user responses to the tests; recording the plurality of user responses; processing the plurality of user responses; and storing the plurality of user responses to compare with a plurality of other recorded user data to determine standards based on user qualifications.

In at least one embodiment of the method, the method steps further include utilizing a saccades vision testing and training module configured to execute on the computing device; displaying a standardized font set at a standardized distance to display a few paragraphs of text at a specified visual angle to a user; detecting a motion of at least one eye of the user in a vertical and a horizontal plane; recording a plurality of eye movements of the at least one eye; processing the recorded eye movements to determine a plurality of features of the eye movements; and storing the recorded eye movements to compare with a plurality of other recorded user data to determine standards based on user qualifications.

In at least one embodiment of the method, the method steps further include utilizing a visual acuity vision testing and training module configured to execute on the computing device, the visual acuity vision testing module when executed: displaying at a standardized distance a test data set to comprising a plurality of visual acuity tests and optotypes to a user; detecting a plurality of user responses, vocal or virtual, to the visual acuity tests; recording the plurality of user responses; processing the plurality of user responses; and storing the plurality of user responses to compare with a plurality of other recorded user data to determine standards based on user qualifications.

In at least one embodiment of the method, the method steps further include utilizing a gross field vision testing and training module configured to execute on the computing device, the gross field vision testing module when executed: displaying at a standardized distance at least one gross field test to a user; detecting a user response, vocal or virtual, to the gross field test; recording the user response; processing the user response; forwarding, if the gross field test result is a fail, the gross field result to indicate a full field test is recommended; and storing the user response to compare with a plurality of other recorded user data to determine standards based on user qualifications.

In at least one embodiment of the method, the method steps further include utilizing a depth perception vision testing and training module configured to execute on the computing device, the depth perception vision testing module when executed: utilizing right eye and left eye projections in space; displaying at a distance of optical infinity and at a reading distance at least one depth perception test to a user; detecting a user response, vocal or virtual, to the depth perception vision test; recording the user response; processing the user response; and storing the user response to compare with a plurality of other recorded user data to determine standards based on user qualifications.

In at least one embodiment of the method, the method steps further include utilizing a color vision testing and training module configured to execute on the computing device; utilizing a plurality of color test projections; displaying at a standardized distance at least one color vision test to a user; detecting a user response, vocal or virtual, to the color vision test; recording the user response; processing the user response; and storing the user response to compare with a plurality of other recorded user data to determine standards based on user qualifications.

In at least one embodiment of the method, the method steps further include utilizing a speed vision testing and training module configured to execute on the computing device; utilizing a plurality of speed reading tests; displaying at a standardized distance at least one speed vision test to a user; detecting a user response, vocal or virtual, to the speed vision test; recording the user response; processing the user response; and storing the user response to compare with a plurality of other recorded user data to determine standards based on user qualifications.

In at least one embodiment of the method, the method steps further include utilizing an Amsler grid vision testing and training module configured to execute on the computing device; utilizing an Amsler grid test; displaying at a standardized distance an Amsler grid vision test to a user; detecting a user response, vocal or virtual, to the Amsler grid vision test; recording the user response; processing the user response; and storing the user response to compare with a plurality of other recorded user data to determine standards based on user qualifications.

In at least one embodiment of the method, the method steps further include: utilizing a keratometry vision testing module configured to execute on the computing device; utilizing a keratometry vision test; utilizing a Placido disc image; displaying a Placido disc image to a user; determining the curvature characteristics of the anterior surface of the cornea; recording the curvature characteristics; processing the curvature characteristics; and storing the curvature characteristics to compare with a plurality of other recorded user data to determine standards based on user qualifications.

In at least one embodiment of the method, the method steps further include utilizing a pupillometry vision testing module configured to execute on the computing device, the pupillometry vision testing module when executed: utilizing a pupillometry vision test; displaying a light to a user; checking the pupil size; measuring the pupillary response of the user to the light; recording the pupillary response; processing the pupillary response; and storing the pupillary response to compare with a plurality of other recorded user data to determine standards based on user qualifications.

In at least one embodiment of the method, the method steps further include utilizing a colorimetry vision testing module configured to execute on the computing device, the colorimetry vision testing module when executed: utilizing a colorimetry dynamic and static field vision test; displaying a plurality of colored lights to a user; measuring the response of the user to the plurality of colored lights; recording the response; processing the response; and storing the response to compare with a plurality of other recorded user data to determine standards based on user qualifications.

In another exemplary embodiment, the technology described herein provides a non-transitory computer readable medium for conducting automated vision tests and associated training using artificial intelligence processing on an extended reality (XR) platform having stored thereon, instructions that when executed in a computing system, cause the computing system to perform operations including: utilizing an extended reality headset display device configured to be worn by a user and operated by the user without direct medical professional assistance; utilizing a computing device communicatively coupled to the extended reality headset display device; utilizing a vision testing and training module configured to execute on the computing device; displaying at least one test data set comprising a plurality of vision tests to a user; detecting a plurality of user responses to the tests; recording the plurality of user responses; processing the plurality of user responses; storing the plurality of user responses to compare with a plurality of other recorded user data to determine standards based on user qualifications. Based on user test results with the XR platform, as measured and recorded from the automated vision tests and compared with a database of normative standards, an optometrist or ophthalmologist may determine and recommend that the user engage in prescribed training exercises using this XR platform and/or determine and prescribe that other visual therapies are needed.

In at least one embodiment of the computer readable medium, the operations further include utilizing a saccades vision testing and training module configured to execute on the computing device; displaying a standardized font set at a standardized distance to display a few paragraphs of text at a specified visual angle to a user; detecting a motion of at least one eye of the user in a vertical and a horizontal plane; recording a plurality of eye movements of the at least one eye; processing the recorded eye movements to determine a plurality of features of the eye movements; and storing the recorded eye movements to compare with a plurality of other recorded user data to determine standards based on user qualifications.

In at least one embodiment of the computer readable medium, the operations further include utilizing a visual acuity vision testing and training module configured to execute on the computing device, the visual acuity vision testing module when executed: displaying at a standardized distance a test data set to comprising a plurality of visual acuity tests and optotypes to a user; detecting a plurality of user responses, vocal or virtual, to the visual acuity tests; recording the plurality of user responses; processing the plurality of user responses; and storing the plurality of user responses to compare with a plurality of other recorded user data to determine standards based on user qualifications.

In at least one embodiment of the computer readable medium, the operations further include utilizing a gross field vision testing and training module configured to execute on the computing device, the gross field vision testing module when executed: displaying at a standardized distance at least one gross field test to a user; detecting a user response, vocal or virtual, to the gross field test; recording the user response; processing the user response; forwarding, if the gross field test result is a fail, the gross field result to indicate a full field test is recommended; and storing the user response to compare with a plurality of other recorded user data to determine standards based on user qualifications.

In at least one embodiment of the computer readable medium, the operations further include utilizing a depth perception vision testing and training module configured to execute on the computing device, the depth perception vision testing module when executed: utilizing right eye and left eye projections in space; displaying at a distance of optical infinity and at a reading distance at least one depth perception test to a user; detecting a user response, vocal or virtual, to the depth perception vision test; recording the user response; processing the user response; and storing the user response to compare with a plurality of other recorded user data to determine standards based on user qualifications.

In at least one embodiment of the computer readable medium, the operations further include utilizing a color vision testing and training module configured to execute on the computing device, the gross field vision testing module when executed: utilizing a plurality of color test projections; displays at a standardized distance at least one color vision test to a user; detecting a user response, vocal or virtual, to the color vision test; recording the user response; processing the user response; and storing the user response to compare with a plurality of other recorded user data to determine standards based on user qualifications.

In at least one embodiment of the computer readable medium, the operations further include utilizing a speed vision testing and training module configured to execute on the computing device; utilizing a plurality of speed reading tests; displaying at a standardized distance at least one speed vision test to a user; detecting a user response, vocal or virtual, to the speed vision test; recording the user response; processing the user response; and storing the user response to compare with a plurality of other recorded user data to determine standards based on user qualifications.

In at least one embodiment of the computer readable medium, the operations further include utilizing an Amsler grid vision testing and training module configured to execute on the computing device; utilizing an Amsler grid test; displaying at a standardized distance an Amsler grid vision test to a user; detecting a user response, vocal or virtual, to the Amsler grid vision test; recording the user response; processing the user response; and storing the user response to compare with a plurality of other recorded user data to determine standards based on user qualifications.

In at least one embodiment of the computer readable medium, the operations further include utilizing a keratometry vision testing module configured to execute on the computing device; utilizing a keratometry vision test; utilizing a Placido disc image; displaying a Placido disc image to a user; determining the curvature characteristics of the anterior surface of the cornea; recording the curvature characteristics; processing the curvature characteristics; and storing the curvature characteristics to compare with a plurality of other recorded user data to determine standards based on user qualifications.

In at least one embodiment of the computer readable medium, the operations further include utilizing a pupillometry vision testing module configured to execute on the computing device, the pupillometry vision testing module when executed: utilizing a pupillometry vision test; displaying a light to a user; checking the pupil size; measuring the pupillary response of the user to the light; recording the pupillary response; processing the pupillary response; and storing the pupillary response to compare with a plurality of other recorded user data to determine standards based on user qualifications.

In at least one embodiment of the computer readable medium, the operations further include utilizing a colorimetry vision testing module configured to execute on the computing device, the colorimetry vision testing module when executed: utilizing a colorimetry dynamic and static field vision test; displaying a plurality of colored lights to a user; measuring the response of the user to the plurality of colored lights; recording the response; processing the response; and storing the response to compare with a plurality of other recorded user data to determine standards based on user qualifications.

Thus, advantageously, the technology described herein provides methods, systems, and devices for the testing of human subjects for a multiplicity of vision tests. Advantageously, the technology described herein provides an automated virtual assistant and eye-movement recording device with extended reality, augmented reality, and virtual reality platforms for automated vision tests of saccades/pursuits, visual acuity, fixations, regressions, depth perception, convergence, divergence, color tests, speed, Amsler grid, keratometry, pupillometry, colorimetry, and other field tests. Advantageously, the technology described herein provides testing and assessment devices, extended reality, augmented reality, and virtual reality goggles, headsets, motion-sensing cameras, and vision training devices. The technology described herein provides many advantages and features over the known systems and methods.

There has thus been outlined, rather broadly, the more important features of the technology in order that the detailed description thereof that follows may be better understood, and in order that the present contribution to the art may be better appreciated. There are additional features of the technology that will be described hereinafter, and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a flowchart diagram depicting a method and various method steps for the testing of human subjects for a multiplicity of vision tests with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

FIG. 2 is a flowchart diagram depicting a method and various method steps for the testing of human subjects for a multiplicity of vision tests with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

FIG. 3 is a schematic diagram depicting a system testing a subject having smart goggles with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

FIG. 4 is a block diagram illustrating the general components of a computer according to an exemplary embodiment of the technology.

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides methods, systems, and devices for the testing of human subjects for a multiplicity of vision tests. More specifically, the technology described herein provides an automated virtual assistant and eye-movement recording device with extended reality, augmented reality, and virtual reality platforms for automated vision tests of saccades/pursuits, visual acuity, fixations, regressions, depth perception, convergence, divergence, color tests, speed, Amsler grid, keratometry, pupillometry, colorimetry, and other field tests. Furthermore, the technology described herein provides testing and assessment devices, extended reality, augmented reality, and virtual reality goggles, headsets, motion-sensing cameras, and vision training devices.

In one exemplary embodiment, the technology described herein provides a system 300 for conducting automated vision tests and associated training using artificial intelligence processing on an extended reality (XR) platform. Based on user test results with the XR platform, as measured and recorded from the automated vision tests and compared with a database of normative standards, an optometrist or ophthalmologist may determine and recommend that the user engage in prescribed training exercises using this XR platform and/or determine and prescribe that other visual therapies are needed.

The term extended reality (XR) will be used throughout. Extended Reality (XR) refers to all real-and-virtual environments generated by computer graphics and wearables. The ‘X’ in XR is simply a variable that can stand for any letter. XR is the umbrella category that covers all the various forms of computer-altered reality, including: Augmented Reality (AR), Mixed Reality (MR), and Virtual Reality (VR).

VR encompasses all virtually immersive experiences. These may be created using real-world content (360 video), purely synthetic content (computer generated), or both. VR requires the use of a Head-Mounted Device (HMD) like the Oculus Rift, HTC Vive, or Google Cardboard.

Augmented Reality (AR) is an overlay of computer-generated content on the real world. The augmented content does not recognize the physical objects within a real-world environment. In other words, the CG content and the real-world content are not able to respond to one another.

Mixed Reality (MR) removes the boundaries between real and virtual interactions via occlusion. Occlusion means the computer-generated objects can be visibly obscured by objects in the physical environment.

The system 300 includes an extended reality headset display device 316 configured to be worn by a user and operated by the user 310 without direct medical professional assistance.

By way of example, the XR headset display device 316 includes goggles, headsets, motion-sensing cameras, and vision training devices. Microsoft provides HoloLens, which is a headset of virtual reality that has transparent lenses that provide an augmented reality experience. The headset in many ways resembles elements of goggles, a cycling helmet, and a welding mask or visor. A user is enabled to view 3D holographic images that appear to be part of an environment. Oculus by Facebook is another VR system available. Oculus has Quest and Rift VR products.

The system 300 includes a computing device 400 communicatively coupled to the extended reality headset display device 316.

The system 300 includes at least one vision testing and training module 318 configured to execute on the computing device 400. The vision testing module 318 when executed: displays at least one test data set comprising a plurality of vision tests to a user; detects a plurality of user responses to the tests; records the plurality of user responses; processes the plurality of user responses; and stores the plurality of user responses to compare with a plurality of other recorded user data to determine standards based on user qualifications.

In at least one embodiment of the system 300, the vision testing and training module further includes a saccades vision testing and training module 340 configured to execute on the computing device 400. The saccades vision testing module 340 when executed: displays a standardized font set at a standardized distance to display a few paragraphs of text at a specified visual angle to a user; detects a motion of at least one eye of the user in a vertical and a horizontal plane; records a plurality of eye movements of the at least one eye; processes the recorded eye movements to determine a plurality of features of the eye movements; and stores the recorded eye movements to compare with a plurality of other recorded user data to determine standards based on user qualifications.

By way of example, in at least one embodiment of the saccades vision testing module 340 the system 300 and the extended reality headset display device 316 will project a standardized font set at a standardized distance to display a few paragraphs at a specified visual angle. The size of the visual angle will be set based on the age of the patient being tested. Appropriate fonts and visual angles are standardized for age groups. The test will use cameras 420 to detect the motion of the eyes in the vertical and horizontal planes. The movements will be recorded and the data of these recordings will be processed by software to determine many features of the eye movements such as length of saccades, number of saccades, time of fixations, number of fixations, regressions, period of regressions, length of regressions, span of perception (number of letters between saccades), convergence and divergence of the eyes, vertical changes between the eyes, return sweeps periods and length and reading rate. Other mathematical findings not mentioned may be determined from the data. A data base of these findings will be kept among patients to determine standards of these findings based on age or other qualifications. The reading material may be in any language and may even consist of random symbols or letters for training or diagnostic purposes. The devices may be used as a diagnostic determination of saccadic functions and then reused for modifying reading habits to make scanning and reading more efficient. Based on user test results on saccades with the XR platform, as measured and recorded from the automated saccades vision test and compared with a database of normative standards, an optometrist or ophthalmologist may determine and recommend that the user engage in prescribed training exercises using this XR platform and/or determine and prescribe that other visual therapies are needed.

Advantageously, the extended reality headset display device 316 allows for control of the distance and visual angle exactly. Also, the AR allows the patient to experience reading in a normal visual space unlike recorders which do not allow peripheral vision or suffer from proximal convergence.

Also, advantageously, the devices may also add or reduce horizontal and/or vertical prismatic demand while reading to determine reading efficiency as well as duction ranges. This may also be used in training sessions for improving aspects of scanning and saccadic functions. Such training might display one word or several words at a time for increasing reading speed.

In at least one embodiment of the system 300, the vision testing and training module 318 further includes a visual acuity vision testing and training module 342 configured to execute on the computing device. The visual acuity vision testing module 342 when executed: displays at a standardized distance a test data set to comprising a plurality of visual acuity tests and optotypes to a user; detects a plurality of user responses, vocal or virtual, to the visual acuity tests; records the plurality of user responses; processes the plurality of user responses; and stores the plurality of user responses to compare with a plurality of other recorded user data to determine standards based on user qualifications. Based on user test results on visual acuity with the XR platform, as measured and recorded from the automated visual acuity vision test and compared with a database of normative standards, an optometrist or ophthalmologist may determine and recommend that the user engage in prescribed training exercises using this XR platform and/or determine and prescribe that other visual therapies are needed.

The visual acuity vision testing and training module 342 includes an automated mode responding to the vocal or virtual responses of the user/patient 310. The user 310 may call out the letters or the user 310 may point to a larger letter from a group projected to the side.

The visual acuity vision testing and training module 342 also provides that instead of letters, children may be tested with Landolt “C” which asks which direction is the open part of the “C”. The Landolt C, also known as a Landolt ring, Landolt broken ring, or Japanese vision test, is an optotype: a standardized symbol used for testing vision. The Landolt C consists of a ring that has a gap, thus looking similar to the letter C. The gap can be at various positions (usually left, right, bottom, top and the 45° positions in between) and the task of the tested person is to decide on which side the gap is. The size of the C and its gap are reduced until the subject makes a specified rate of errors. The minimum perceivable angle of the gap is taken as measure of the visual acuity.

The visual acuity vision testing and training module 342 also provides that dynamic visual acuities used in sports vision could also be tested where the chart moves during testing or the head is made to move while testing by making the patient keep their head pointing toward the moving projected bar. The visual acuity vision testing and training module 342 also provides that rotation trainers, such as those depicted at https://www.bernell.com/productaWRG/Rotation-Trainers may be displayed.

In at least one embodiment of the system 300, the vision testing and training module 318 further includes a gross field vision testing and training module 344 configured to execute on the computing device 400. The gross field vision testing module 344 when executed: displays at a standardized distance at least one gross field test to a user; detects a user response, vocal or virtual, to the gross field test; records the user response; processes the user response; forwards, if the gross field test result is a fail, the gross field result to indicate a full field test is recommended; and stores the user response to compare with a plurality of other recorded user data to determine standards based on user qualifications.

The gross field vision testing and training module 344 is configured to test for “gross confrontations.” For example, traditionally, in an in-person exam, a doctor will say “look at my nose.” The doctor will hold one hand on the left and one on the right of the patient. “Tell me how many fingers I am holding out.” Now the doc does the same up and down and then diagonally. The gross field vision testing and training module 344 is configured to conduct a similar automated test like this looking not for a field test, but a gross field test. If the user 310 misses one, the need for a real test is indicated.

In at least one embodiment of the system 300, the vision testing and training module 318 further includes a depth perception vision testing and training module 346 configured to execute on the computing device 400. The depth perception vision testing module 346 when executed: utilizes right eye and left eye projections in space; displays at a distance of optical infinity and at a reading distance at least one depth perception test to a user; detects a user response, vocal or virtual, to the depth perception vision test; records the user response; processes the user response; and stores the user response to compare with a plurality of other recorded user data to determine standards based on user qualifications. The distance of optical infinity is, by way of example and not of limitation, typically twenty feet. The reading distance, by way of example and not of limitation, is forty centimeters.

The depth perception vision testing and training module 346 is configured to use standard right eye and left eye projections in space. By way of example Wirt circles are used such as those depicted at https://www.bernell.com/product/SOM150/Depth-Perception-Tests. The depth perception vision testing and training module 346 is configured to use two objects in space like a Howard-Dolman Type Test such as those depicted at https://www.bernell.com/product/HDTEST/Depth-Perception-Tests. The depth perception vision testing and training module 346 is configured to use random dot patterns projected at different distances such as those depicted at https://www.bernell.com/product/VA1015/Depth-Perception-Tests.

In at least one embodiment of the system 300, the vision testing and training module 318 further includes a color vision testing and training module 348 configured to execute on the computing device 400. The color vision testing module 348 when executed: utilizes a plurality of color test projections; displays at a standardized distance at least one color vision test to a user; detects a user response, vocal or virtual, to the color vision test; records the user response; processes the user response; and stores the user response to compare with a plurality of other recorded user data to determine standards based on user qualifications. Based on user test results on color with the XR platform, as measured and recorded from the automated color vision test and compared with a database of normative standards, an optometrist or ophthalmologist may determine and recommend that the user engage in prescribed training exercises using this XR platform and/or determine and prescribe that other visual therapies are needed. For example, colorblind persons are not generally blind to color (this is the real exception). Most are anomalous. They see a weaker color than others. Most red/green “colorblind” men can actually tell the two apart except when they are desaturated too much. They can be trained or become experienced enough to improve their skill, but not necessarily ever reach normal.

The color vision testing and training module 348 is configured to use an Ishihara type test for color blindness such as those depicted at https://www.bernell.com/product/CVT1/Color_Vision_Test_Books. The color vision testing and training module 348 is configured to use the Farnsworth D15 Color Test such as those depicted at https://www.bernell.com/product/LF15PC/Farnsworthand other Farnsworth tests. BY way of example, the D15 or D100 tests are moved in front of the user, and the user manipulates the virtual discs in space.

In at least one embodiment of the system 300, the vision testing and training module 318 further includes a speed vision testing and training module 350 configured to execute on the computing device 400. The speed vision testing module 350 when executed: utilizes a plurality of speed reading tests; displays at a standardized distance at least one speed vision test to a user; detects a user response, vocal or virtual, to the speed vision test; records the user response; processes the user response; and stores the user response to compare with a plurality of other recorded user data to determine standards based on user qualifications.

The speed vision testing and training module 350 is configured for training to improve reading by increasing the speed of the words shown; showing the words as wider and wider fixations of words or by auditory penalizing of the patient/user 310 when the recorder detects a regression. The speed vision testing and training module 350 is configured to show, for example, a gray paragraph and darken a word or parts of words and then darken words or parts of words to the right and lighten the word or parts of words to the left so as to make it appear the darkening is moving. The reader is expected to “keep up” with the words which are darker. This could also be done with color changes or with changing the location of a background rectangle to make it appear to be moving. One might also flash the increase in darkness of the words to make motion appear or flash the words or portions of words themselves to train fixation as well as widening the span of fixation.

In at least one embodiment of the system 300, the vision testing and training module 318 further includes an Amsler grid vision testing and training module 352 configured to execute on the computing device 400. The Amsler grid vision testing module 352 when executed: utilizes an Amsler grid test; displays at a standardized distance an Amsler grid vision test to a user; detects a user response, vocal or virtual, to the Amsler grid vision test; records the user response; processes the user response; and stores the user response to compare with a plurality of other recorded user data to determine standards based on user qualifications.

The Amsler grid vision testing and training module 352 is configured to conduct grid testing at near as well as at distances to detect potential field loss or distortions caused by retinal detachments.

In at least one embodiment of the system 300, the vision testing and training module 318 further includes a keratometry vision testing module 354 configured to execute on the computing device 400. The keratometry vision testing module 354 when executed: utilizes a keratometry vision test; utilizes a Placido disc image; displays a Placido disc image to a user; determines the curvature characteristics of the anterior surface of the cornea; records the curvature characteristics; processes the curvature characteristics; and stores the curvature characteristics to compare with a plurality of other recorded user data to determine standards based on user qualifications.

The keratometry vision testing module 354 is configured to reflect onto the corneas a Placido Disc having concentric rings, such as white rings on a black background. As such, the test can determine the curvature of the corneas.

In at least one embodiment of the system 300, the vision testing and training module 318 further includes a pupillometry vision testing module 356 configured to execute on the computing device 400. The pupillometry vision testing module 356 when executed: utilizes a pupillometry vision test; displays a light to a user; checks the pupil size; measures the pupillary response of the user to the light; records the pupillary response; processes the pupillary response; and stores the pupillary response to compare with a plurality of other recorded user data to determine standards based on user qualifications.

By way of example, the pupillometry vision testing module 356 is configured to measure the speed of pupillary response. The pupillometry vision testing module 356 may be used as a sideline test for concussions. The pupillometry vision testing module 356 may be used as a swinging flashlight test. The pupillometry vision testing module 356 may be used in the detection of neurological disorders such as Parkinson's or Alzheimer's.

In at least one embodiment of the system 300, the vision testing and training module 318 further includes a colorimetry vision testing module 350 configured to execute on the computing device 400. The colorimetry vision testing module 358 when executed: utilizes a colorimetry dynamic and static field vision test; displays a plurality of colored lights to a user; measures the response of the user to the plurality of colored lights; records the response; processes the response; and stores the response to compare with a plurality of other recorded user data to determine standards based on user qualifications.

The colorimetry vision testing module 358 is configured to provide a dynamic and static field test done using different colors. Colored lights cause fields to be expanded or contracted depending on the parasympathetic/sympathetic balance of the patient. This is not at all the same thing as field testing as these colored field tests may differ by 50% depending on the wavelength of light while regular fields vary 2-5% per test.

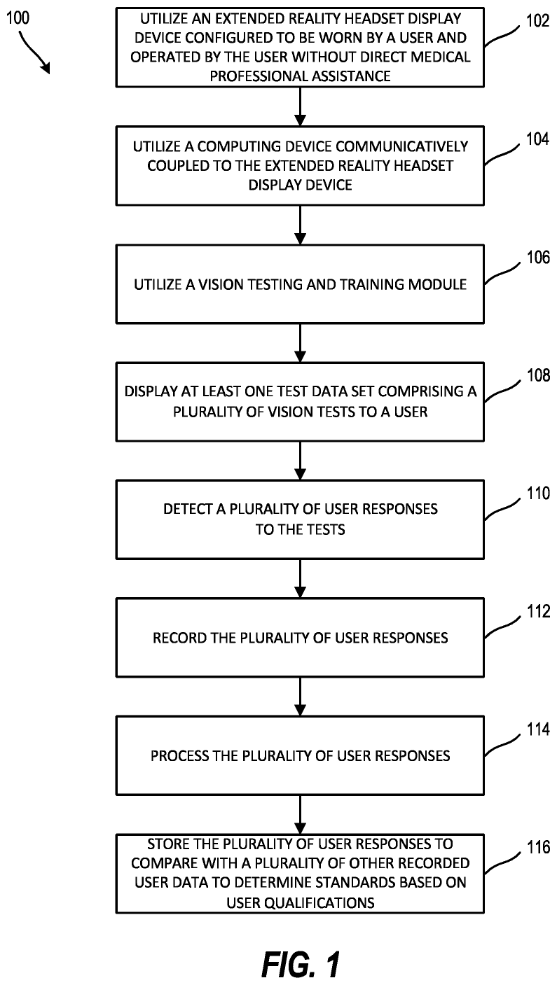

Referring now to FIG. 1, a flowchart diagram 100 depicting a method and various method steps for the testing of human subjects for a multiplicity of vision tests with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

At step 102, an extended reality headset display device is utilized. The extended reality headset display device configured to be worn by a user and operated by the user without direct medical professional assistance.

At step 104, a computing device is utilized. The computing device is communicatively coupled to the extended reality headset display device.

At step 106, a vision testing and training module is utilized.

At step 108, at least one test data set is displayed. The data set includes a plurality of vision tests to a user.

At step 110, a plurality of user responses to the tests is detected.

At step 112, the plurality of user responses is recorded.

At step 114, the plurality of user responses is processed.

At step 116, the plurality of user responses is stored and then compared with a plurality of other recorded user data to determine standards based on user qualifications.

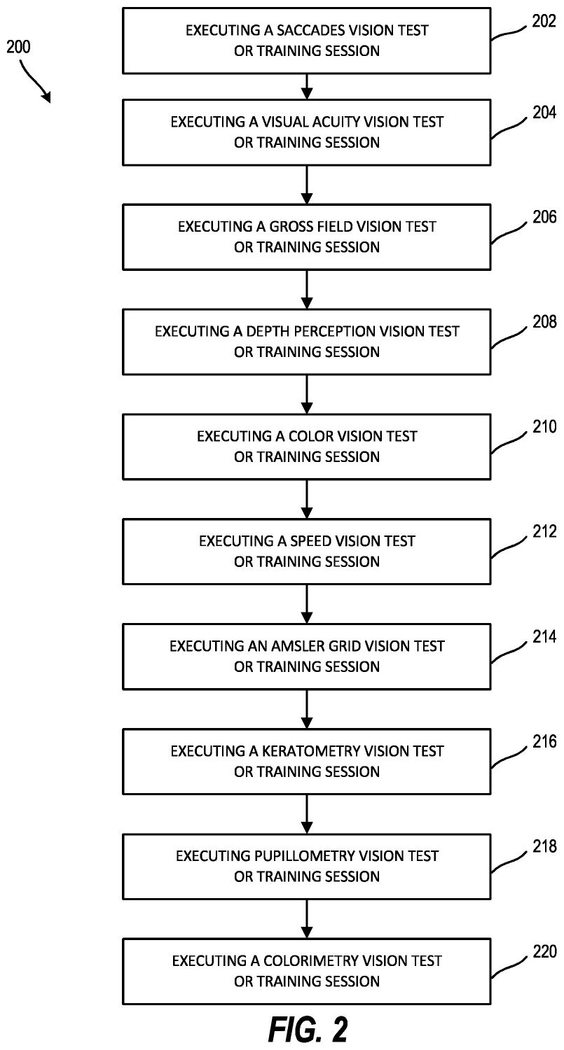

Referring now to FIG. 2, a flowchart diagram 200 depicting additional, various method steps for the testing of human subjects for a multiplicity of vision tests with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

At step 202, a saccades vision test or training session is executed.

At step 204, a visual acuity vision test or training session is executed.

At step 206, a gross field vision test or training session is executed.

At step 208, a depth perception vision test or training session is executed.

At step 210, a color vision test or training session is executed.

At step 212, a speed vision test or training session is executed.

At step 214, an Amsler grid vision test or training session is executed.

At step 216, a keratometry vision test or training session is executed.

At step 218, a pupillometry vision test or training session is executed.

At step 220, a colorimetry vision test or training session is executed.

The method steps depicted in FIGS. 1 and 2 do not necessarily occur sequentially and may vary as determined by a test administrator or user 310. Additionally, not all methods steps listed are required, as may be determined by a test administer. The steps listed are exemplary and may be varied in both order and selection.

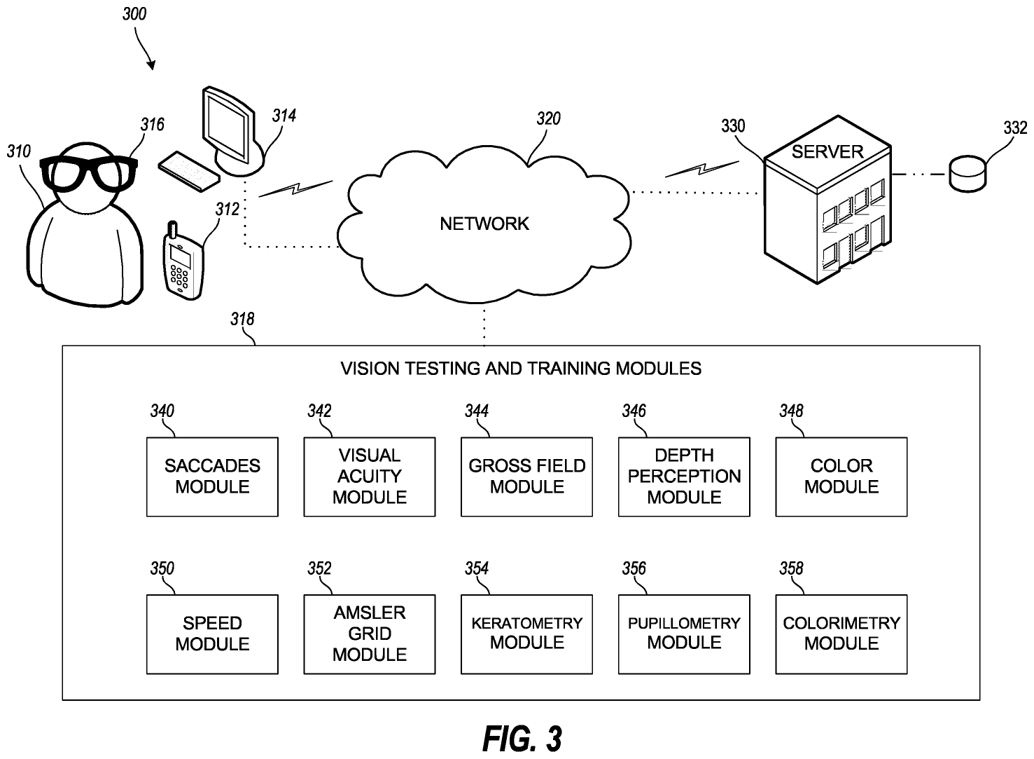

FIG. 3 is a schematic diagram 300 depicting a system testing a subject having smart goggles with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests, according to an embodiment of the technology described herein.

The test subject/patient 310 may utilize an extended reality device such as XR goggles 316 to assess the vision testing and training module 318 and thereby conduct vision tests and/or vision training exercises. Additional devices such as a computer 314 or a smart device 312 may be utilized by an administrator for additional support and/or connectivity. The extended reality device such as XR goggles 316 is coupled to a network 320, such as the public internet, and is cloud based on at least one embodiment. The extended reality device such as XR goggles 316 can access one or more remote servers 330 for the processing and or storing of data and utilize one or more databases 332 in network-based implementations.

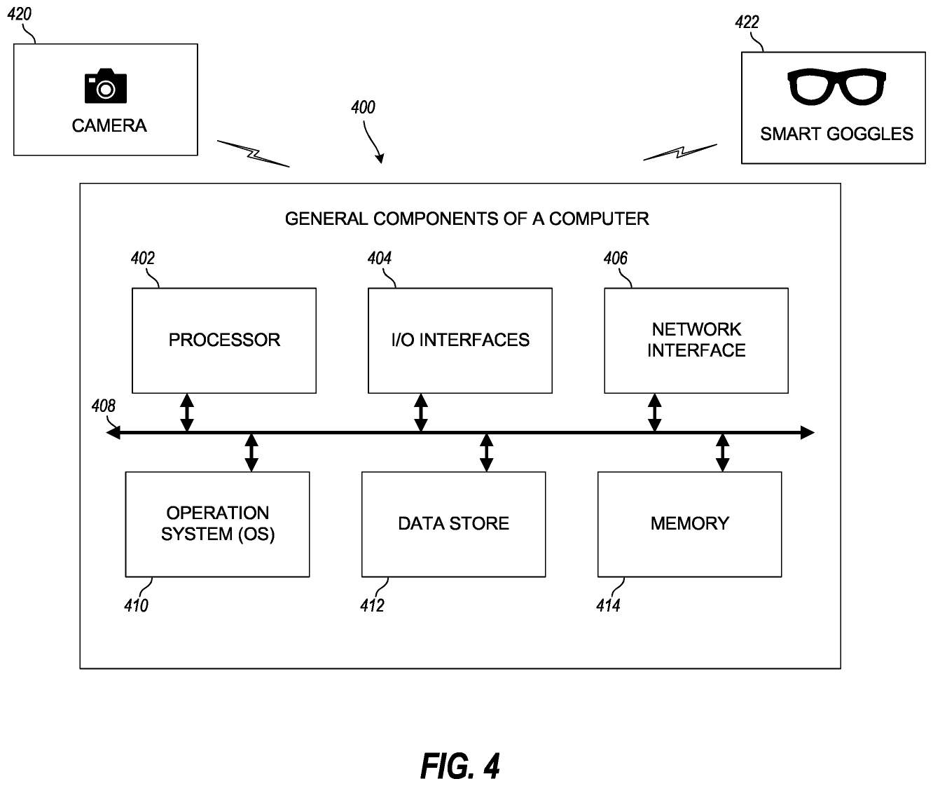

Referring now to FIG. 4, a block diagram 400 illustrating the general components of a computer is shown. Any one or more of the computers, servers, database, and the like, disclosed above, may be implemented with such hardware and software components. The computer 400 can be a digital computer that, in terms of hardware architecture, generally includes a processor 402, input/output (I/O) interfaces 404, network interfaces 406, an operating system (O/S) 410, a data store 412, and a memory 414. The components (402, 404, 406, 410, 412, and 414) are communicatively coupled via a local interface 408. The local interface 408 can be, for example but not limited to, one or more buses or other wired or wireless connections, as is known in the art. The local interface 408 can have additional elements, which are omitted for simplicity, such as controllers, buffers (caches), drivers, among many others, to enable communications. Further, the local interface 408 can include address, control, and/or data connections to enable appropriate communications among the aforementioned components. The general operation of a computer comprising these elements is well known in the art.

In various embodiments, the components 400 also include, or are integrally formed with, smart goggles 422, XR headsets, and XR accessories, and with cameras and recorders 420.

The processor 402 is a hardware device for executing software instructions. The processor 402 can be any custom made or commercially available processor, a central processing unit (CPU), an auxiliary processor among several processors associated with the computer 400, a semiconductor-based microprocessor (in the form of a microchip or chip set), or generally any device for executing software instructions. When the computer 400 is in operation, the processor 402 is configured to execute software stored within the memory 414, to communicate data to and from the memory 414, and to generally control operations of the computer 400 pursuant to the software instructions.

The I/O interfaces 404 can be used to receive user input from and/or for providing system output to one or more devices or components. User input can be provided via, for example, a keyboard and/or a mouse, or smart device such as googles or XR equipment. System output can be provided via a display device and a printer (not shown). I/O interfaces 404 can include, for example but not limited to, a serial port, a parallel port, a small computer system interface (SCSI), an infrared (IR) interface, a radio frequency (RF) interface, and/or a universal serial bus (USB) interface.

The network interfaces 406 can be used to enable the computer 400 to communicate on a network. For example, the computer 400 can utilize the network interfaces 408 to communicate via the internet to other computers or servers for software updates, technical support, etc. The network interfaces 408 can include, for example, an Ethernet card (e.g., 10BaseT, Fast Ethernet, Gigabit Ethernet) or a wireless local area network (WLAN) card (e.g., 802.11a/b/g). The network interfaces 408 can include address, control, and/or data connections to enable appropriate communications on the network.

A data store 412 can be used to store data, such as information regarding positions entered in a requisition. The data store 412 can include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 412 can incorporate electronic, magnetic, optical, and/or other types of storage media. In one example, the data store 412 can be located internal to the computer 400 such as, for example, an internal hard drive connected to the local interface 408 in the computer 400. Additionally, in another embodiment, the data store can be located external to the computer 400 such as, for example, an external hard drive connected to the I/O interfaces 404 (e.g., SCSI or USB connection). Finally, in a third embodiment, the data store may be connected to the computer 400 through a network, such as, for example, a network attached file server.

The memory 414 can include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 414 may incorporate electronic, magnetic, optical, and/or other types of storage media. Note that the memory 414 can have a distributed architecture, where various components are situated remotely from one another, but can be accessed by the processor 402.

The software in memory 414 can include one or more software programs, each of which includes an ordered listing of executable instructions for implementing logical functions. In the example of FIG. 4, the software in the memory system 414 includes the interactive toolkit for sourcing valuation and a suitable operating system (O/S) 410. The operating system 410 essentially controls the execution of other computer programs, such as the interactive toolkit for sourcing valuation, and provides scheduling, input-output control, file and data management, memory management, and communication control and related services. The operating system 410 can be any of Windows NT, Windows 2000, Windows XP, Windows Vista, Windows 7, 8, 10 (all available from Microsoft, Corp. of Redmond, Wash.), Solaris (available from Sun Microsystems, Inc. of Palo Alto, Calif.), LINUX (or another UNIX variant) (available from Red Hat of Raleigh, N.C.), Chrome OS by Google, or other like operating system with similar functionality.

In an exemplary embodiment of the technology described herein, the computer 400 is configured to perform flowcharts 100 and 200 depicted in FIGS. 1 and 2 respectively to enable user vision testing and training with a method and various method steps for the testing of human subjects for a multiplicity of vision tests with an automated virtual assistant and eye-movement recording device with extended reality, augmented reality, and virtual reality platforms for automated vision tests of saccades/pursuits, visual acuity, fixations, regressions, depth perception, convergence, divergence, color tests, and other field tests.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. A system for conducting automated vision tests and associated training using artificial intelligence processing on an extended reality (XR) platform, the system comprising:
an extended reality headset display device configured to be worn by a user and operated by the user without direct medical professional assistance;
a computing device communicatively coupled to the extended reality headset display device;
a vision testing and training module configured to execute on the computing device, the vision testing module when executed:
displays at least one test data set comprising a plurality of vision tests to a user;
detects a plurality of user responses to the tests;
records the plurality of user responses;
processes the plurality of user responses; and
stores the plurality of user responses to compare with a plurality of other recorded user data to determine standards based on user qualifications.

2. The system for conducting automated vision tests and associated training of claim 1, wherein the vision testing and training module further comprises:
a saccades vision testing and training module configured to execute on the computing device, the saccades vision testing module when executed:
displays a standardized font set at a standardized distance to display a few paragraphs of text at a specified visual angle to a user;
detects a motion of at least one eye of the user in a vertical and a horizontal plane;
records a plurality of eye movements of the at least one eye;
processes the recorded eye movements to determine a plurality of features of the eye movements; and
stores the recorded eye movements to compare with a plurality of other recorded user data to determine standards based on user qualifications.

3. The system for conducting automated vision tests of claim 1, wherein the vision testing and training module further comprises:
a visual acuity vision testing and training module configured to execute on the computing device, the visual acuity vision testing module when executed:
displays at a standardized distance a test data set to comprising a plurality of visual acuity tests and optotypes to a user;
detects a plurality of user responses, vocal or virtual, to the visual acuity tests;
records the plurality of user responses;
processes the plurality of user responses; and
stores the plurality of user responses to compare with a plurality of other recorded user data to determine standards based on user qualifications.

4. The system for conducting automated vision tests of claim 1, wherein the vision testing and training module further comprises:
a gross field vision testing and training module configured to execute on the computing device, the gross field vision testing module when executed:
displays at a standardized distance at least one gross field test to a user;
detects a user response, vocal or virtual, to the gross field test;
records the user response;
processes the user response;
if the gross field test result is a fail, forwards the gross field result to indicate a full field test is recommended; and
stores the user response to compare with a plurality of other recorded user data to determine standards based on user qualifications.

5. The system for conducting automated vision tests of claim 1, wherein the vision testing and training module further comprises:
a depth perception vision testing and training module configured to execute on the computing device, the depth perception vision testing module when executed:
utilizes right eye and left eye projections in space;
displays at a distance of optical infinity and at a reading distance at least one depth perception test to a user;
detects a user response, vocal or virtual, to the depth perception vision test;
records the user response;
processes the user response; and
stores the user response to compare with a plurality of other recorded user data to determine standards based on user qualifications.

6. The system for conducting automated vision tests of claim 1, wherein the vision testing and training module further comprises:
a color vision testing and training module configured to execute on the computing device, the gross field vision testing module when executed:
utilizes a plurality of color test projections;
displays at a standardized distance at least one color vision test to a user;
detects a user response, vocal or virtual, to the color vision test;
records the user response;
processes the user response; and
stores the user response to compare with a plurality of other recorded user data to determine standards based on user qualifications.

7. The system for conducting automated vision tests of claim 1, wherein the vision testing and training module further comprises:
a speed vision testing and training module configured to execute on the computing device, the speed vision testing module when executed:
utilizes a plurality of speed reading tests;
displays at a standardized distance at least one speed vision test to a user;
detects a user response, vocal or virtual, to the speed vision test;
records the user response;
processes the user response; and
stores the user response to compare with a plurality of other recorded user data to determine standards based on user qualifications.

8. The system for conducting automated vision tests of claim 1, wherein the vision testing and training module further comprises:
an Amsler grid vision testing and training module configured to execute on the computing device, the Amsler grid vision testing module when executed:
utilizes an Amsler grid test;
displays at a standardized distance an Amsler grid vision test to a user;
detects a user response, vocal or virtual, to the Amsler grid vision test;
records the user response;
processes the user response; and
stores the user response to compare with a plurality of other recorded user data to determine standards based on user qualifications.

9. The system for conducting automated vision tests of claim 1, wherein the vision testing and training module further comprises:
a keratometry vision testing module configured to execute on the computing device, the keratometry vision testing module when executed:
utilizes a keratometry vision test;
utilizes a Placido disc image;
displays a Placido disc image to a user;
determines the curvature characteristics of the anterior surface of the cornea;
records the curvature characteristics;
processes the curvature characteristics; and
stores the curvature characteristics to compare with a plurality of other recorded user data to determine standards based on user qualifications.

10. The system for conducting automated vision tests of claim 1, wherein the vision testing and training module further comprises:
a pupillometry vision testing module configured to execute on the computing device, the pupillometry vision testing module when executed:
utilizes a pupillometry vision test;
displays a light to a user;
checks the pupil size;
measures the pupillary response of the user to the light;
records the pupillary response;
processes the pupillary response; and
stores the pupillary response to compare with a plurality of other recorded user data to determine standards based on user qualifications.

11. The system for conducting automated vision tests of claim 1, wherein the vision testing and training module further comprises:
a colorimetry vision testing module configured to execute on the computing device, the colorimetry vision testing module when executed:
utilizes a colorimetry dynamic and static field vision test;
displays a plurality of colored lights to a user;
measures the response of the user to the plurality of colored lights;
records the response;
processes the response; and
stores the response to compare with a plurality of other recorded user data to determine standards based on user qualifications.

12. A method for conducting automated vision tests and associated training using artificial intelligence processing on an extended reality (XR) platform, the method comprising:
utilizing an extended reality headset display device configured to be worn by a user and operated by the user without direct medical professional assistance;
utilizing a computing device communicatively coupled to the extended reality headset display device;
utilizing a vision testing and training module configured to execute on the computing device;
displaying at least one test data set comprising a plurality of vision tests to a user;
detecting a plurality of user responses to the tests;
recording the plurality of user responses;
processing the plurality of user responses; and
storing the plurality of user responses to compare with a plurality of other recorded user data to determine standards based on user qualifications.

13. The method for conducting automated vision tests of claim 12, further comprising:
utilizing a saccades vision testing and training module configured to execute on the computing device;
displaying a standardized font set at a standardized distance to display a few paragraphs of text at a specified visual angle to a user;
detecting a motion of at least one eye of the user in a vertical and a horizontal plane;
recording a plurality of eye movements of the at least one eye;
processing the recorded eye movements to determine a plurality of features of the eye movements; and
storing the recorded eye movements to compare with a plurality of other recorded user data to determine standards based on user qualifications.

14. The method for conducting automated vision tests of claim 12, further comprising:
utilizing a visual acuity vision testing and training module configured to execute on the computing device, the visual acuity vision testing module when executed:
displaying at a standardized distance a test data set to comprising a plurality of visual acuity tests and optotypes to a user;
detecting a plurality of user responses, vocal or virtual, to the visual acuity tests;
recording the plurality of user responses;
processing the plurality of user responses; and
storing the plurality of user responses to compare with a plurality of other recorded user data to determine standards based on user qualifications.

15. The method for conducting automated vision tests of claim 12, further comprising:
utilizing a gross field vision testing and training module configured to execute on the computing device, the gross field vision testing module when executed:
displaying at a standardized distance at least one gross field test to a user;
detecting a user response, vocal or virtual, to the gross field test;
recording the user response;
processing the user response;
forwarding, if the gross field test result is a fail, the gross field result to indicate a full field test is recommended; and
storing the user response to compare with a plurality of other recorded user data to determine standards based on user qualifications.

16. The method for conducting automated vision tests of claim 12, further comprising:
utilizing a depth perception vision testing and training module configured to execute on the computing device, the depth perception vision testing module when executed:
utilizing right eye and left eye projections in space;
displays at a distance of optical infinity and at a reading distance at least one depth perception test to a user;
detecting a user response, vocal or virtual, to the depth perception vision test;
recording the user response;
processing the user response; and
storing the user response to compare with a plurality of other recorded user data to determine standards based on user qualifications.

17. The method for conducting automated vision tests of claim 12, further comprising:
utilizing a color vision testing and training module configured to execute on the computing device;
utilizing a plurality of color test projections;
displaying at a standardized distance at least one color vision test to a user;
detecting a user response, vocal or virtual, to the color vision test;
recording the user response;
processing the user response; and
storing the user response to compare with a plurality of other recorded user data to determine standards based on user qualifications.

18. The method for conducting automated vision tests of claim 12, further comprising:
utilizing a speed vision testing and training module configured to execute on the computing device;
utilizing a plurality of speed reading tests;
displaying at a standardized distance at least one speed vision test to a user;
detecting a user response, vocal or virtual, to the speed vision test;
recording the user response;
processing the user response; and
storing the user response to compare with a plurality of other recorded user data to determine standards based on user qualifications.

19. The method for conducting automated vision tests of claim 12, further comprising:
utilizing an Amsler grid vision testing and training module configured to execute on the computing device;
utilizing an Amsler grid test;
displaying at a standardized distance an Amsler grid vision test to a user;
detecting a user response, vocal or virtual, to the Amsler grid vision test;
recording the user response;
processing the user response; and
storing the user response to compare with a plurality of other recorded user data to determine standards based on user qualifications.

20. The method for conducting automated vision tests of claim 12, further comprising:
utilizing an keratometry vision testing module configured to execute on the computing device;
utilizing a keratometry vision test;
utilizing a Placido disc image;
displaying a Placido disc image to a user;
determining the curvature characteristics of the anterior surface of the cornea;
recording the curvature characteristics;
processing the curvature characteristics; and
storing the curvature characteristics to compare with a plurality of other recorded user data to determine standards based on user qualifications.

21. The method for conducting automated vision tests of claim 12, further comprising:
utilizing a pupillometry vision testing module configured to execute on the computing device, the pupillometry vision testing module when executed:
utilizing a pupillometry vision test;
displaying a light to a user;
checking the pupil size;
measuring the pupillary response of the user to the light;
recording the pupillary response;
processing the pupillary response; and
storing the pupillary response to compare with a plurality of other recorded user data to determine standards based on user qualifications.

22. The method for conducting automated vision tests of claim 12, further comprising:
utilizing a colorimetry vision testing module configured to execute on the computing device, the colorimetry vision testing module when executed:
utilizing a colorimetry dynamic and static field vision test;
displaying a plurality of colored lights to a user;
measuring the response of the user to the plurality of colored lights;
recording the response;
processing the response; and
storing the response to compare with a plurality of other recorded user data to determine standards based on user qualifications.

23. A non-transitory computer readable medium for conducting automated vision tests and associated training using artificial intelligence processing on an extended reality (XR) platform having stored thereon, instructions that when executed in a computing system, cause the computing system to perform operations comprising:
utilizing an extended reality headset display device configured to be worn by a user and operated by the user without direct medical professional assistance;
utilizing a computing device communicatively coupled to the extended reality headset display device;
utilizing a vision testing and training module configured to execute on the computing device;
displaying at least one test data set comprising a plurality of vision tests to a user;
detecting a plurality of user responses to the tests;
recording the plurality of user responses;
processing the plurality of user responses; and
storing the plurality of user responses to compare with a plurality of other recorded user data to determine standards based on user qualifications.

24. The computer readable medium of claim 23, wherein the instructions that when executed in a computing system, cause the computing system to perform the additional operations comprising:
utilizing a saccades vision testing and training module configured to execute on the computing device;
displaying a standardized font set at a standardized distance to display a few paragraphs of text at a specified visual angle to a user;
detecting a motion of at least one eye of the user in a vertical and a horizontal plane;
recording a plurality of eye movements of the at least one eye;
processing the recorded eye movements to determine a plurality of features of the eye movements; and
storing the recorded eye movements to compare with a plurality of other recorded user data to determine standards based on user qualifications.

25. The computer readable medium of claim 23, wherein the instructions that when executed in a computing system, cause the computing system to perform the additional operations comprising:
utilizing a visual acuity vision testing and training module configured to execute on the computing device, the visual acuity vision testing module when executed:
displaying at a standardized distance a test data set to comprising a plurality of visual acuity tests and optotypes to a user;
detecting a plurality of user responses, vocal or virtual, to the visual acuity tests;
recording the plurality of user responses;
processing the plurality of user responses; and
storing the plurality of user responses to compare with a plurality of other recorded user data to determine standards based on user qualifications.

26. The computer readable medium of claim 23, wherein the instructions that when executed in a computing system, cause the computing system to perform the additional operations comprising:
utilizing a gross field vision testing and training module configured to execute on the computing device, the gross field vision testing module when executed:
displaying at a standardized distance at least one gross field test to a user;
detecting a user response, vocal or virtual, to the gross field test;
recording the user response;
processing the user response;
forwarding, if the gross field test result is a fail, the gross field result to indicate a full field test is recommended; and
storing the user response to compare with a plurality of other recorded user data to determine standards based on user qualifications.

27. The computer readable medium of claim 23, wherein the instructions that when executed in a computing system, cause the computing system to perform the additional operations comprising:
utilizing a depth perception vision testing and training module configured to execute on the computing device, the depth perception vision testing module when executed:
utilizing right eye and left eye projections in space;
displays at a distance of optical infinity and at a reading distance at least one depth perception test to a user;
detecting a user response, vocal or virtual, to the depth perception vision test;
recording the user response;
processing the user response; and
storing the user response to compare with a plurality of other recorded user data to determine standards based on user qualifications.

28. The computer readable medium of claim 23, wherein the instructions that when executed in a computing system, cause the computing system to perform the additional operations comprising:
utilizing a color vision testing and training module configured to execute on the computing device, the gross field vision testing module when executed:
utilizing a plurality of color test projections;
displays at a standardized distance at least one color vision test to a user;
detecting a user response, vocal or virtual, to the color vision test;
recording the user response;
processing the user response; and
storing the user response to compare with a plurality of other recorded user data to determine standards based on user qualifications.

29. The computer readable medium of claim 23, wherein the instructions that when executed in a computing system, cause the computing system to perform the additional operations comprising:
utilizing a speed vision testing and training module configured to execute on the computing device’
utilizing a plurality of speed reading tests;
displaying at a standardized distance at least one speed vision test to a user;
detecting a user response, vocal or virtual, to the speed vision test;
recording the user response;
processing the user response; and
storing the user response to compare with a plurality of other recorded user data to determine standards based on user qualifications.

30. The computer readable medium of claim 23, wherein the instructions that when executed in a computing system, cause the computing system to perform the additional operations comprising:
utilizing an Amsler grid vision testing and training module configured to execute on the computing device;
utilizing an Amsler grid test;
displaying at a standardized distance an Amsler grid vision test to a user;
detecting a user response, vocal or virtual, to the Amsler grid vision test;
recording the user response;
processing the user response; and
storing the user response to compare with a plurality of other recorded user data to determine standards based on user qualifications.

31. The computer readable medium of claim 23, wherein the instructions that when executed in a computing system, cause the computing system to perform the additional operations comprising:
utilizing an keratometry vision testing module configured to execute on the computing device;
utilizing a keratometry vision test;
utilizing a placido disc image;
displaying a placido disc image to a user;
determining the curvature characteristics of the anterior surface of the cornea;
recording the curvature characteristics;
processing the curvature characteristics; and
storing the curvature characteristics to compare with a plurality of other recorded user data to determine standards based on user qualifications.

32. The computer readable medium of claim 23, wherein the instructions that when executed in a computing system, cause the computing system to perform the additional operations comprising:
utilizing a pupillometry vision testing module configured to execute on the computing device, the pupillometry vision testing module when executed:
utilizing a pupillometry vision test;
displaying a light to a user;
checking the pupil size;
measuring the pupillary response of the user to the light;
recording the pupillary response;
processing the pupillary response; and
storing the pupillary response to compare with a plurality of other recorded user data to determine standards based on user qualifications.

33. The computer readable medium of claim 23, wherein the instructions that when executed in a computing system, cause the computing system to perform the additional operations comprising:
utilizing a colorimetry vision testing module configured to execute on the computing device, the colorimetry vision testing module when executed:
utilizing a colorimetry dynamic and static field vision test;
displaying a plurality of colored lights to a user;
measuring the response of the user to the plurality of colored lights;
recording the response;
processing the response; and
storing the response to compare with a plurality of other recorded user data to determine standards based on user qualifications.
